(19) United States
(12) Patent Application Publication
(10) Pub. No.: US 2025/0213750 A1
(43) Pub. Date: Jul. 3, 2025

(54) PARTICLE-CONTAINING HYDROGEL

(71) Applicant: SEKISUI KASEI CO., LTD., Osaka (JP)

(72) Inventor: Eisaku SATO, Osaka (JP)

(73) Assignee: SEKISUI KASEI CO., LTD., Osaka (JP)

(21) Appl. No.: 18/851,797

(22) PCT Filed: Feb. 17, 2023
(86) PCT No.: PCT/JP2023/005845

§ 371 Date: Sep. 27, 2024

(30) Foreign Application Priority Data
Mar. 29, 2022 (JP) 2022-052962

(51) Int. Cl. A61L 27/52; A61L 15/42; A61L 15/60; A61L 27/14; A61L 27/54; A61L 27/56; C08K 5/00

(52) U.S. Cl. CPC A61L 27/52; A61L 15/425; A61L 15/60; A61L 27/14; A61L 27/54; A61L 27/56; C08K 5/00

(57) ABSTRACT

An object of the present invention is to provide a hydrogel capable of stably dispersing a large amount of volatile component and excellent in sustained release of volatile component. Provided is a hydrogel comprising a porous resin particle and a volatile component, the porous resin particle having a pore diameter of 5 to 30 nm and an oil absorption of 50 to 700 ml/100 g, and a mass ratio between the volatile component (A) and the porous resin particle (B) contained in the hydrogel being (A):(B)=1:1 to 1:4.

### TECHNICAL FIELD

The present invention relates to a hydrogel.

### BACKGROUND ART

Hydrogels are obtained by swelling a polymer having high affinity with water in an aqueous solvent. Hydrogels have various properties such as water absorbency, swellability, moisture retention, adhesion, and conductivity depending on their applications, and are used in a wide range of fields such as civil engineering and construction, agriculture, food, medicine, cosmetics, and electricity by utilizing these properties.

For example, PTL 1 discloses a hydrogel containing a polymer matrix and water and having a specific network structure.

Also, a perfume may be used in hydrogels as an additive, but when menthol is used, it is difficult to dissolve menthol in a mixture liquid for preparing hydrogels since menthol is crystalline at room temperature, and there is a problem that when a large amount of menthol is blended, the mixture liquid and the hydrogel prepared from the mixture liquid become cloudy. PTL 2 discloses a hydrogel containing 0.001 to 1% by mass of 1-menthol, but when 1-menthol is added in an amount exceeding 1% by mass, the hydrogel becomes cloudy, and it is difficult to prepare a hydrogel with an increased content of 1-menthol.

### CITATION LIST

### Patent Literature

PTL 1: JP2018-150458A
PTL 2: JP2021-147543A

### SUMMARY OF INVENTION

### Technical Problem

An object of the present invention is to provide a hydrogel capable of stably dispersing a large amount of volatile component and excellent in sustained release of volatile component.

### Solution to Problem

In order to solve the above problems, the inventors have extensively conducted studies, and resultantly found that a large amount of volatile component can be stably dispersed in a hydrogel by encapsulating the volatile component in porous resin particles and dispersing the porous resin particles in the hydrogel, and the hydrogel is excellent in sustained release of volatile component.

The present invention has been completed based on these findings, and includes inventions of the following broad aspects.

### Item 1

A hydrogel comprising a porous resin particle and a volatile component,
the porous resin particle having a pore diameter of 5 to 30 nm and an oil absorption of 50 to 700 ml/100 g, and
a mass ratio between the volatile component (A) and the porous resin particle (B) contained in the hydrogel being
(A):(B)=1:1 to 1:4.

### Item 2

The hydrogel according to item 1, in which the porous resin particle comprises a monofunctional (meth)acrylic acid ester and a crosslinkable monomer.

### Item 3

The hydrogel according to item 1 or 2, in which a content of the volatile component is 0.5 to 5% by mass based on 100% by mass of the hydrogel.

### Item 4

The hydrogel according to any one of items 1 to 3, in which the volatile component is at least one selected from the group consisting of perfumes, spices, and essential oils.

### Item 5

The hydrogel according to any one of items 1 to 4, comprising a polymer matrix, water, and a wetting agent.

### Item 6

The hydrogel according to item 5, in which the polymer matrix comprises a copolymer of a monofunctional monomer having one ethylenically unsaturated group, and a crosslinkable monomer.

### Item 7

The hydrogel according to item 6, in which a content of the structural unit derived from the monofunctional monomer is 10 to 40% by mass based on 100% by mass of the hydrogel.

### Item 8

The hydrogel according to item 6 or 7, in which the monofunctional monomer is at least one selected from the group consisting of (meth)acrylamide, (meth)acrylic acid, N,N-dimethyl(meth)acrylamide, diacetone (meth)acrylamide, and tert-butyl acrylamide sulfonic acid.

### Item 9

The hydrogel according to any one of items 5 to 8, in which a content of the wetting agent is 20 to 70% by mass based on 100% by mass of the hydrogel.

### Item 10

The hydrogel according to any one of items 1 to 9, which is used as a biological electrode in a monitoring device or a device that performs treatment using electric stimulation, a counter electrode plate for an electrosurgical device, an adhesive tape, or a wound covering material.

### Advantageous Effects of Invention

According to the present invention, it is possible to provide a hydrogel capable of stably dispersing a large amount of volatile component and excellent in sustained release of volatile component.

### BRIEF DESCRIPTION OF DRAWINGS

FIG. 1A is a schematic plan view of an embodiment of a gel sheet.

FIG. 1B is a schematic cross-sectional view taken along line 1B-1B of the gel sheet of FIG. 1A.

### DESCRIPTION OF EMBODIMENTS

As used herein, the singular forms (a, an, the, etc.) are intended to include singular and plural forms, unless the context indicates otherwise or clearly dictates otherwise.

In the present specification, “comprise” is a concept also including “consist essentially of” and “consist of”.

In the numerical range described in stages in the present specification, the upper or lower limit of the numerical range at one stage can be optionally combined with the upper or lower limit of the numerical range at another stage. Also, in the numerical range described in the present specification, the upper or lower limit of the numerical range may be replaced with a value shown in Examples or a value that can be uniquely derived from Examples. Further, in the present specification, the numerical values connected by “to” mean the numerical range including the numerical values before and after “to” as the lower limit and the upper limit.

In the present specification, “(meth)acryl” refers to acryl, or both. Therefore, (meth)acrylamide and/or N,N-dimethyl(meth)acrylamide refers to (1) acrylamide, methacrylamide, or both, (2) N,N-dimethylacrylamide, N,N-dimethylmethacrylamide, or both, or (3) both of (1) and (2).

### Hydrogel

The hydrogel of the present invention is a hydrogel containing a porous resin particle and a volatile component, the porous resin particle has a pore diameter of 5 to 30 nm and an oil absorption of 50 to 700 ml/100 g, and a mass ratio between the volatile component (A) and the porous resin particle (B) contained in the hydrogel is (A) (B)=1:1 to 1:4.

In the hydrogel of the present invention, the porous resin particle and the volatile component are preferably contained in the hydrogel as a volatile component-containing particle in which a volatile component is supported in the porous resin particle.

### Porous Resin Particle

The porous resin particle is not particularly limited as long as it is a resin particle having a porous structure, but one that is water-insoluble is preferable. The water-insoluble property of the resin particle means not only a property that the resin particle itself is water-insoluble but also a property that the water-soluble acrylic resin, alginic acid resin, or amide resin is crosslinked to be water-insoluble. Here, the term “water-insoluble” refers to a property of not being dissolved but being in a suspended and/or dispersed state at a visual level after being added to water.

Examples of the porous resin particle include an acrylic resin, an alginic acid resin, an amide resin, and the like. Among them, a porous resin particle made of an acrylic resin is preferable.

Examples of the porous resin particle made of an acrylic resin include porous resin particles made of a polymer of a monomer mixture, in which the monomer mixture contains a monofunctional (meth)acrylic acid ester and a crosslinkable monomer as monomers.

Examples of the monofunctional (meth)acrylic acid ester include methyl (meth)acrylate, ethyl (meth)acrylate, propyl (meth)acrylate, butyl (meth)acrylate, pentyl (meth)acrylate, hexyl (meth)acrylate, heptyl (meth)acrylate, octyl (meth)acrylate, nonyl (meth)acrylate, decyl (meth)acrylate, undecyl (meth)acrylate, dodecyl (meth)acrylate, glycidyl (meth)acrylate, methoxyethyl (meth)acrylate, propoxyethyl (meth)acrylate, butoxyethyl (meth)acrylate, methoxydiethyleneglycol (meth)acrylate, ethoxydiethyleneglycol (meth)acrylate, methoxyethyleneglycol (meth)acrylate, butoxytriethyleneglycol (meth)acrylate, methoxydipropyleneglycol (meth)acrylate, phenoxyethyl (meth)acrylate, phenoxydiethyleneglycol (meth)acrylate, phenoxytetraethyleneglycol (meth)acrylate, benzyl (meth)acrylate, cyclohexyl (meth)acrylate, tetrahydrofurfuryl (meth)acrylate, dicyclopentenyl (meth)acrylate, dicyclopentenyloxyethyl (meth)acrylate, N-vinyl-2-pyrrolidone (meth)acrylate, 2-hydroxyethyl (meth)acrylate, hydroxypropyl (meth)acrylate, hydroxybutyl (meth)acrylate, 2-hydroxy-3-phenyloxypropyl (meth)acrylate, and the like. These monofunctional (meth)acrylic acid esters may be used singly or in combination of two or more. As the monofunctional (meth)acrylic acid ester used in the present invention, C1 to C4 (1 to 4 carbon atoms) alkyl ester of (meth)acrylic acid is preferable, and methyl methacrylate is particularly preferable.

The content of the monofunctional (meth)acrylic acid ester in the monomer mixture is within the range of 1 to 50% by mass, and preferably within the range of 10 to 50% by mass. If the content of the monofunctional (meth)acrylic acid ester in the monomer mixture is within the range of 1 to 50% by mass, the crosslinkable monomer can be sufficiently contained in the monomer mixture, so that sufficient porosity can be imparted to the porous resin particle to increase the specific surface area, and the bulk specific gravity of the porous resin particle can be reduced.

As the crosslinkable monomer, a known crosslinkable monomer having two or more ethylenically unsaturated groups can be used.

Examples of the crosslinkable monomer include (meth)acrylic crosslinkable monomers such as ethyleneglycol di(meth)acrylate, 1,6-hexanediol di(meth)acrylate, allyl (meth)acrylate, trimethylolpropane tri(meth)acrylate, and pentaerythritol tetra(meth)acrylate; vinyl-based crosslinkable monomers, that are divinylbenzene, divinylnaphthalene, diallylphthalate, and derivatives thereof; and the like. Among them, a (meth)acrylic crosslinkable monomer is preferable, and ethyleneglycol di(meth)acrylate is more preferable. These crosslinkable monomers may be used singly or in combination of two or more.

In addition, the content of the crosslinkable monomer in the monomer mixture is within the range of 50 to 99% by mass, and more preferably within the range of 50 to 90% by mass. If the content of the crosslinkable monomer in the monomer mixture is within the range of 50 to 99% by mass, sufficient porosity can be imparted to the porous resin particle to increase the specific surface area, and the bulk specific gravity of the porous resin particle can be reduced.

Also, the monomer mixture may contain monomers other than the monofunctional (meth)acrylic acid ester and the crosslinkable monomer as long as the specific surface area of the porous resin particle of the present invention is not affected.

The porous resin particle has a pore diameter of 5 to 30 nm, and preferably 10 to 20 nm. If the average pore diameter is less than 5 nm, permeation of volatile component into the porous resin particle may be inhibited. In addition, if the pore diameter exceeds 30 nm, the release rate of volatile component from the porous resin particle is increased, and the sustained release of the volatile component in the hydrogel may be deteriorated. The pore diameter means an average pore diameter obtained based on BJH method from nitrogen desorption isotherm. As a measurement method by the BJH method, for example, the nitrogen desorption isotherm is measured for resin particles to be measured using a specific surface area/pore distribution measuring device (Tristar II 3020, manufactured by Shimadzu Corporation), and a pore diameter (average pore diameter) can be calculated based on the BJH method (Barrett, E. P.; Joyner, L. G.; Halenda, P. P., J. Am. Chem. Soc. 73, 373 (1951)).

The porous resin particle has an oil absorption of 50 to 700 ml/100 g, and preferably 100 to 700 ml/100 g. If the oil absorption is 50 ml/100 g or more, a large amount of volatile component can be supported in the porous resin particle, which is preferable. If the oil absorption exceeds 700 ml/100 g, voids inside the porous resin particle increase, and there is a possibility that sufficient particle strength cannot be secured. The oil absorption can be measured based on the measurement method of JIS K 5101-13-2.

The porous resin particle has a pore volume of preferably 0.30 to 0.90 ml/g, and more preferably 0.40 to 0.70 ml/g. If the pore volume is 0.30 ml/g or more, a large amount of volatile component can be supported in the porous resin particle, which is preferable. Also, if the pore volume is 0.90 ml/g or less, the particle strength of the porous resin particle does not decrease, which is preferable. In the present specification, the pore volume refers to a pore volume per unit mass, and in the present invention, means a pore volume obtained from the nitrogen desorption isotherm using the BJH method. As a measurement method by the BJH method, for example, the nitrogen desorption isotherm is measured for resin particles to be measured using a specific surface area/pore distribution measuring device (Tristar II 3020, manufactured by Shimadzu Corporation), and a pore volume (integrated pore volume) can be calculated based on the BJH method (Barrett, E. P.; Joyner, L. G.; Halenda, P. P., J. Am. Chem. Soc. 73, 373 (1951)).

The porous resin particle has a specific surface area of preferably 50 to 300 m²/g, and more preferably 80 to 200 m²/g. If the specific surface area is 50 m²/g or more, a large amount of volatile component can be absorbed in the porous resin particle, which is preferable. If the specific surface area is 300 m²/g or less, the particle strength of the porous resin particle does not decrease, which is preferable. In the present specification, the specific surface area refers to a surface area per unit mass, and in the present invention, means a specific surface area obtained by the BET method (N₂). The measurement of the specific surface area by the BET method (N₂) can be performed according to the BET method (nitrogen adsorption method) described in ISO 9277, first edition, JIS Z 8830: 2001. For example, the BET nitrogen adsorption isotherm is measured for resin particles to be measured using a specific surface area/pore distribution measuring device (Tristar II 3020, manufactured by Shimadzu Corporation), and the specific surface area can be calculated from the nitrogen adsorption amount using BET multipoint method.

The porous resin particle has a bulk specific gravity of preferably 0.20 to 0.70 g/ml, and more preferably 0.30 to 0.60 g/ml. If the bulk specific gravity is 0.20 g/ml or more, the particle strength of the porous resin particle does not decrease, which is preferable. If the bulk specific gravity is 0.70 g/ml or less, a large amount of volatile component can be supported in the porous resin particle, which is preferable. In the present specification, the bulk specific gravity means a solid apparent specific gravity measured using Powder Tester PT-E type manufactured by Hosokawa Micron Corporation.

The porous resin particle has a volume average particle diameter of preferably within the range of 0.50 to 100 μm, more preferably within the range of 1.0 to 50 μm, and particularly preferably within the range of 2.0 to 30 μm. When the volume average particle diameter is within the above range, the permeation of volatile component into the porous resin particle can be effectively exhibited. In the measurement of the volume average particle diameter, 0.1 g of resin particles are dispersed in 10 ml of a 0.1% by mass aqueous solution of a nonionic surfactant using a particle size distribution measuring device (Multisizer 4e (manufactured by Beckman Coulter, Inc.), and the volume average particle diameter is measured using the dispersion. The volume average particle diameter is calculated from an average value in a volume-based particle size distribution of 100,000 particles.

The porous resin particle can be produced by subjecting a monomer mixture containing a monofunctional (meth)acrylic acid ester and a crosslinkable monomer to suspension polymerization in an aqueous medium in the presence of an organic solvent, and then removing the organic solvent.

The content of the porous resin particle in the hydrogel is preferably 0.50 to 10% by mass and more preferably 1.0 to 6.0% by mass based on 100% by mass of the hydrogel. When the content of the porous resin particle is within the above range, a large amount of volatile component can be added to the hydrogel without impairing the mechanical strength and flexibility of the hydrogel.

### Volatile Component

Examples of the volatile component include perfumes, spices, essential oils, and the like, and these can be used singly or as a mixture of two or more.

Examples of the perfumes and spices include citrus essential oils such as 1-menthol, orange oil, lemon oil, grapefruit oil, lime oil, tangerine oil, lavender oil, mandarin oil, and bergamot oil; spice oils of sage, rosemary, perilla, basil, ginger, horseradish, and the like; oleoresins obtained by solvent extraction of these; aromatic vegetable oil such as coffee oil, roast nut oils, and sesame oil; natural or synthetic perfume compounds such as vanillin, maltol, linalool, geraniol, citral, and limonene; and the like. From the viewpoint of effects, l-menthol is preferable, and as the l-menthol, in addition to natural menthol and synthetic menthol, menthol-containing essential oils such as mint oil, peppermint oil, and spearmint oil can be used singly or in combination.

Examples of the essential oil include white cedar essential oil, Japanese cypress essential oil, Japanese cedar essential oil, pine essential oil, and the like.

The content of the volatile component in the hydrogel is preferably 0.50 to 5.0% by mass and more preferably 1.0 to 3.0% by mass based on 100% by mass of the hydrogel. If the content of the volatile component is 0.50% by mass or more, a hydrogel excellent in sustained release of volatile component is obtained, which is preferable. If the content of the volatile component is 5.0% by mass or less, the volatile component hardly precipitates in the hydrogel, which is preferable.

In addition, the volatile component can be optionally used as a solvent solution diluted with a solvent. The solvent is not particularly limited as long as it is uniformly mixed with the volatile component, but ester oil, silicone oil, hydrocarbon oil, waxes, and the like are preferable. Also, as commercially available products, NEOLIGHT 100P (manufactured by Kokyu Alcohol Kogyo Co., Ltd.), KAK HL (manufactured by Kokyu Alcohol Kogyo Co., Ltd.), and the like are preferable.

When a solvent solution containing a volatile component is used, the solvent solution is preferably prepared so that the concentration of the volatile component in the solvent solution is 20% by mass or more.

Furthermore, the content of the solvent in the hydrogel is preferably 0 to 5.0% by mass based on 100% by mass of the hydrogel. When the content of the solvent is within the above range, a large amount of volatile component can be supported in the porous resin particle, and a hydrogel excellent in sustained release of volatile component can be obtained, which is preferable.

### Method for Preparing Porous Resin Particle Supporting Volatile Component (Volatile Component-Containing Particle)

A porous resin particle supported with a volatile component (volatile component-containing particle) can be obtained by mixing and stirring a volatile component or a solvent solution containing a volatile component and a porous resin particle. The stirring time is not particularly limited, but is about 0.50 to 12 hours.

In the present invention, the mass ratio particle the volatile component (A) and the porous resin particle (B) ((A):(B)) in the volatile component-containing particle contained in the hydrogel is 1:1 to 1:4, and preferably 1:2 to 1:2.5. Within the above range, a volatile component can be supported in the porous particle, and a hydrogel excellent in sustained release of volatile component can be obtained.

In addition, the hydrogel of the present invention preferably contains a polymer matrix, water, and a wetting agent.

### Polymer Matrix

The polymer matrix is contained in an amount of preferably 10 to 40% by mass and more preferably 15 to 30% by mass in 100% by mass of the hydrogel. If the content is 10% by mass or more, the hydrogel has sufficient shape retention, and there is little possibility that the hydrogel is too soft or easily torn. Also, if the content is 40% by mass or less, the flexibility of the hydrogel is hardly impaired.

The polymer matrix is not particularly limited as long as it forms a network structure and can form a gel containing at least water. For example, the polymer matrix can be formed from a copolymer of a monofunctional monomer having one ethylenically unsaturated group, and a crosslinkable monomer.

### (1) Monofunctional Monomer

The monofunctional monomer is not particularly limited as long as it has one ethylenically unsaturated group, but is preferably a water-soluble monomer. Examples of the monofunctional monomer include alkyl (meth)acrylamides such as (meth)acrylamide, dialkyl(meth)acrylamides such as N,N-dimethyl(meth)acrylamide and N,N-diethyl (meth)acrylamide, N-isopropyl (meth)acrylamide, N-methyl (meth)acrylamide, N-ethyl(meth)acrylamide, and N-propyl (meth)acrylamide; hydroxyalkyl (meth)acrylamides such as N-hydroxyethyl (meth)acrylamide and N-hydroxymethyl (meth)acrylamide; alkoxyalkyl (meth)acrylamides such as N-ethoxymethyl (meth)acrylamide, N-propoxymethyl (meth)acrylamide, N-butoxymethyl (meth)acrylamide, N-isobutoxymethyl (meth)acrylamide, N-pentoxymethyl (meth)acrylamide, N-hexyloxymethyl (meth)acrylamide, N-heptoxymethyl (meth)acrylamide, N-octoxymethyl (meth)acrylamide, N-ethoxyethyl (meth)acrylamide, N-propoxyethyl (meth)acrylamide, and N-butoxyethyl (meth)acrylamide; amino group-containing cationic acrylamide-based compounds such as N,N-dimethylaminopropyl(meth)acrylamide; sulfonic acid group-containing anionic monofunctional monomers such as 4-acryloylmorpholine and tert-butyl acrylamide sulfonic acid or salts thereof; diacetone (meth)acrylamide; (meth)acrylic acid or salts thereof; derivatives thereof; and the like. Among them, at least one selected from the group consisting of (meth)acrylamide, N,N-dimethyl(meth)acrylamide, diacetone (meth)acrylamide, tert-butylacrylamide sulfonic acid and salts thereof, and (meth)acrylic acid and salts thereof is preferably used, but it is not limited thereto. These monofunctional monomers may be used singly or in a combination of two or more.

Further, as the monofunctional monomer, in addition to the above, optionally, a vinylamide monofunctional monomer such as N-vinylpyrrolidone, N-vinylacetamide or N-vinylformamide; a nonionic monofunctional monomer such as allyl alcohol, a styrene monomer, or the like can be used. These monofunctional monomers may be used singly or in a combination of two or more.

The content of the structural unit derived from the monofunctional monomer in the hydrogel is preferably in the range of 10 to 40% by mass and more preferably 15 to 35% by mass based on 100% by mass of the hydrogel. If the content of the structural unit derived from the monofunctional monomer is within the above range, it is preferable from the viewpoint of the shape, adhesive force, handleability, and flexibility of the hydrogel. If the content is 10% by mass or more, deterioration of shape stability due to a small monofunctional monomer amount hardly occurs, and a cohesive force and a holding force of the hydrogel itself are not reduced, so that a hydrogel having an appropriate adhesive force is obtained. Also, if the content is 40% by mass or less, a hydrogel having an appropriate adhesive force is obtained, and the flexibility of the hydrogel is hardly impaired.

### (2) Crosslinkable Monomer

As the crosslinkable monomer, it is preferable to use a monomer having two or more double bonds having polymerizability in the molecule. Examples of such a crosslinkable monomer include polyfunctional (meth)acrylamides or (meth)acrylates, such as N,N′-methylenebis(meth)acrylamide, N,N′-ethylenebis(meth)acrylamide, (poly)ethyleneglycol di(meth)acrylate, (poly)propyleneglycol di(meth)acrylate, (poly)glycerol di(meth)acrylate, and (poly)glycerol tri(meth)acrylate; tetraallyloxyethane; diallyl ammonium chloride; and the like, and these can be used alone or in a combination of two or more. As the crosslinkable monomer having two or more double bonds having polymerizability in the molecule, a polyglycerol derivative that is a polyfunctional compound having two or more (meth)acryloyl groups or vinyl groups and having a molecular weight of 400 or more, described in JP2803886B1, can also be used.

The content of the structural unit derived from the crosslinkable monomer in the hydrogel is preferably within the range of 0.010% by mass to 0.50% by mass and more preferably 0.010% by mass to 0.10% by mass based on 100% by mass of the hydrogel. If the content of the structural unit derived from the crosslinkable monomer is within the above range, it is preferable from the viewpoint of the shape, adhesive force, handleability, and flexibility of the hydrogel. If the content is 0.010% by mass or more, deterioration of shape stability due to a low crosslinking density hardly occurs, and a cohesive force and a holding force of the hydrogel itself are not reduced, so that a hydrogel having an appropriate adhesive force is obtained. In addition, deterioration of handleability of the gel sheet, for example, a part of the gel material remaining on the adherend at the time of peeling, also hardly occurs. When the content is 0.50% by mass or less, a hydrogel having an appropriate adhesive force is obtained, and the flexibility of the hydrogel is hardly impaired.

### Water

The content of water in the hydrogel is not particularly limited, but is preferably 10 to 60% by mass and more preferably 15 to 30% by mass based on 100% by mass of the hydrogel. If the content of water is 10% by mass or more, the water content with respect to the equilibrium water content of the hydrogel is not too low, and deterioration derived from hygroscopicity (for example, swelling or the like) of the hydrogel hardly occurs. In addition, if the content of water is 60% by mass or less, the water content with respect to the equilibrium water content of the hydrogel is not too large, and deterioration derived from drying (for example, contraction or the like) of the hydrogel hardly occurs.

### Wetting Agent

The wetting agent is not particularly limited, and examples thereof include diols, such as ethylene glycol, triethylene glycol, 1,6-hexanediol, 1,9-nonanediol, propylene glycol, and butanediol; tri- or higher polyhydric alcohols, such as glycerol, pentaerythritol, and sorbitol; polyhydric alcohol condensates, such as polyethylene glycol, polypropylene glycol, and polyglycerol; modified polyhydric alcohols, such as polyoxyethylene glycerol; polyoxyethylene alkyl ethers, such as polyoxyethylene lauryl ether, polyoxyethylene stearyl ether, polyoxyethylene isostearyl ether, and polyoxyethylene methyl glucoside, polyoxyalkylene alkyl ethers, such as polyoxypropylene alkyl ethers, such as polyoxypropylene lauryl ether, polyoxypropylene stearyl ether, polyoxypropylene isostearyl ether, and polyoxypropylene methyl glucoside; monosaccharides, such as xylose, arabinose, glucose, galactose, and mannose; disaccharides, such as sucrose, maltose, cellobiose, and lactose; oligosaccharides such as maltotriose, polysaccharides, such as xylan, starch, cellulose, chitin, and chitosan; and the like. Amino sugars of these saccharides and N-acetylated products thereof are also applicable. These may be used in combination of any two or more thereof, regardless of D-form or L-form.

Among the wetting agents, it is preferable to use a polyhydric alcohol that is liquid in a use temperature range of the hydrogel (for example, around 20° C. when used indoors), and specifically, ethylene glycol, triethylene glycol, propylene glycol, polypropylene glycol, polyethylene glycol, polyglycerol, glycerol, and the like are suitable.

The content of the wetting agent in the hydrogel is not particularly limited, but is preferably within the range of 20 to 70% by mass and more preferably within the range of 30 to 60% by mass based on 100% by mass of the hydrogel. If the content of the wetting agent is 20% by mass or more, the hydrogel to be obtained has a moisturizing power, and evaporation of moisture is also suppressed, so that temporal stability of the hydrogel is improved, which is preferable. In addition, if the content of the wetting agent is 70% by mass or less, the wetting agent does not bleed out from the hydrogel surface, and a decrease in adhesive force due to the bleed out is suppressed, which is preferable.

### Electrolyte

In addition, the hydrogel can optionally contain an electrolyte, which can impart conductivity to the hydrogel.

In the case of imparting conductivity to the hydrogel, the content of the electrolyte in the hydrogel is preferably 0.050 to 10% by mass and more preferably 0.10 to 6.0% by mass based on 100% by mass of the hydrogel. If the content of the electrolyte is 0.050% by mass or more, impedance of the hydrogel sheet decreases, and conductivity is good, which is preferable. On the other hand, if the content of the electrolyte in the hydrogel is too large, it is difficult to dissolve the electrolyte in the hydrogel, and there is a possibility that precipitation of crystals occurs in the gel or dissolution of other components is inhibited. In addition, conductivity reaches a plateau, and further addition cannot be said to be beneficial from the viewpoint of imparting conductivity.

The electrolyte is not particularly limited, and examples thereof include alkali metal halides such as sodium halides (for example, sodium chloride), lithium halides, and potassium halides; alkaline earth halides such as magnesium halides and calcium halides; other metal halides; and the like. In addition, as the electrolyte, hypochlorites, chlorites, chlorates, perchlorates, hydrochlorides, sulfates, carbonates, nitrates, and phosphates of various metals are also suitably used. Moreover, as the electrolyte, inorganic salts, such as ammonium salts and complex salts; salts of monovalent organic carboxylic acids, such as acetic acid, benzoic acid, and lactic acid; salts of polyvalent organic carboxylic acids, such as tartaric acid; monovalent, divalent, or higher valent salts of polyvalent carboxylic acids, such as phthalic acid, succinic acid, adipic acid, and citric acid; metal salts of organic acids, such as sulfonic acids and amino acids; organic ammonium salts; and the like are also preferable.

Further, a base such as sodium hydroxide may be appropriately added to the hydrogel for the purpose of adjusting the pH.

### Additive

Furthermore, the hydrogel may optionally contain other additives. Examples of the other additives include rust inhibitors, antifungal agents, antioxidants, dispersants, antifoaming agents, stabilizers, surfactants, colorants, and the like.

### Intermediate Substrate

The hydrogel of the present invention may further include an intermediate substrate embedded along the in-plane direction. Here, the in-plane direction of the hydrogel indicates an arbitrary direction in a plane orthogonal to the thickness direction of the hydrogel. The presence of the intermediate substrate leads to reinforcement of the hydrogel, improvement in shape retention at the time of cutting, and the like. The hydrogel in which the intermediate substrate is embedded along the in-plane direction can also be represented as a composite material including the hydrogel and the intermediate substrate embedded along the in-plane direction of the hydrogel interchangeably.

The intermediate substrate is not particularly limited, and examples thereof include a nonwoven fabric, a woven fabric, paper, a film, and the like. As the material of the intermediate substrate, natural fibers such as cellulose, silk, and hemp, synthetic fibers such as polyester, nylon, rayon, polyethylene, polypropylene, and polyurethane, or blends thereof can be used, and a binder may be optionally used, and further, coloring may be optionally performed.

The method for producing the nonwoven fabric is not particularly limited, and examples thereof include a dry method, a wet method, a spunbond method, a melt-blown method, an air-laid method, a chemical bond method, a thermal bond method, a needle punch method, and a water-jet interlacing method. For the position control of the intermediate substrate, it is more preferable to adopt a production method depending on the basis weight, the material, and the like so as to have an even basis weight. The woven fabric is also not particularly limited, and examples include a plain weave, tricot, raschel, and the like. The woven fabric can be appropriately selected.

In addition, the basis weight of the nonwoven fabric or the woven fabric is not particularly limited as long as it is the basis weight that enables the fabric to have predetermined physical properties as an intermediate substrate, but is preferably, for example, 10 to 100 g/m². If the basis weight of the nonwoven fabric or woven fabric is 10 g/m² or more, for example, it is possible to reinforce the gel sheet. If the basis weight is 100 g/m² or less, the intermediate substrate is not excessively hard, and the followability of the hydrogel to the skin and the continuity are not impaired.

If the intermediate substrate is too thick, the permeability of a liquid decreases, and conversely, if the intermediate substrate is too thin, for example, it may be impossible to reinforce the gel sheet as in the case where the basis weight is too small. The thickness of the intermediate substrate is thus suitably determined, taking these into consideration. The thickness of the intermediate substrate is preferably within the range of 0.050 mm to 2.0 mm, more preferably 0.050 mm to 0.50 mm, and particularly preferably 0.080 mm to 0.30 mm.

The thickness of the hydrogel of the present invention is appropriately selected according to the application, and is, for example, within the range of 0.20 mm to 2.0 mm. In a preferred embodiment, the thickness of the hydrogel of the present invention is 0.30 mm to 1.2 mm.

### Application of Hydrogel

The hydrogel of the present invention can stably disperse a large amount of volatile component and is excellent in sustained release of volatile component.

The hydrogel of the present invention is excellent in flexibility, water retention, and the like and thus can be used in a wide range of fields such as medical care, cosmetics, food, chemistry, civil engineering, agriculture, bioengineering, and sports. For example, the hydrogel of the present invention can be used as a biological electrode hydrogel, a cooling gel, a cosmetic face mask, a cell culture medium, or the like. Preferably, the hydrogel of the present invention can be used as a biological electrode in a monitoring device or a device that performs treatment using electric stimulation, a counter electrode plate for an electrosurgical device, an adhesive tape, or a wound covering material.

### Method for Producing Hydrogel

The hydrogel according to the embodiment of the present invention is not particularly limited, but is composed of a gelled substance which can be obtained by uniformly dispersing the above-described materials other than water and a polymerization initiator in water, and polymerizing and crosslinking the obtained mixture liquid by, for example, heating or ultraviolet irradiation. The polymerization initiator may be a thermal polymerization initiator or a photopolymerization initiator, and a known thermal polymerization initiator or photopolymerization initiator for polymerizing acrylic monomers can be used. In addition, the content of the polymerization initiator is not particularly limited, but is preferably 0.010% by mass or more and preferably 1.0% by mass or less based on 100% by mass of the total amount of the resulting hydrogel (the total amount of the mixture liquid). Further, when polymerization is performed by ultraviolet irradiation, the integrated amount of ultraviolet irradiation varies also depending on, for example, the content of the polymerization initiator. For example, the integrated amount of ultraviolet irradiation is preferably within the range of 800 to 10,000 mJ/cm², and more preferably within the range of 2,000 to 10,000 mJ/cm². By setting the amount of the polymerization initiator and the amount of ultraviolet irradiation appropriately, the reaction rate of a non-crosslinkable monomer and the crosslinkable monomer can be suitably adjusted.

The hydrogel of the embodiment of the present invention can be formed into a desired shape, such as a sheet, by pouring a mixture liquid into a container having a desired shape, such as a bottomed container having a substantially rectangular cross-section and performing polymerization by, for example, heating or ultraviolet irradiation. The hydrogel formed into a sheet may have any shape according to the purpose. For example, the sheet of the hydrogel may have a substantially rectangular shape or a substantially circular shape, but is not limited thereto. Hereinafter, the hydrogel in the form of a sheet is referred to as a “hydrogel sheet” or simply a “gel sheet”.

The production process of the hydrogel sheet including an intermediate substrate is not particularly limited. The detailed conditions vary depending on, for example, the composition of the hydrogel, the material of the intermediate substrate, and the thickness. For example, a method such as the following can be suitably used: a method in which an intermediate substrate is held in the air with at least a certain degree of tension applied so that the deformation of the intermediate substrate in the vertical direction is minimized, and a mixture liquid is poured onto the upper and lower sides of the intermediate substrate and polymerized by light irradiation or the like to form a sheet; a method in which two sheet-like hydrogels with a smooth surface are prepared, and an intermediate substrate held with at least a certain degree of tension applied is then sandwiched between these hydrogels to form a composite; a method in which a sheet-like hydrogel with a smooth surface is prepared, an intermediate substrate is placed on the hydrogel while at least a certain degree of tension is applied, and a monomer mixture liquid is poured on the intermediate substrate and further polymerized by light irradiation or the like; or the like. When the production process is a continuous process, the intermediate substrate and/or the hydrogel may be formed into rolls, then removed therefrom, and suitably cut into sheets.

### Embodiment of Hydrogel Sheet

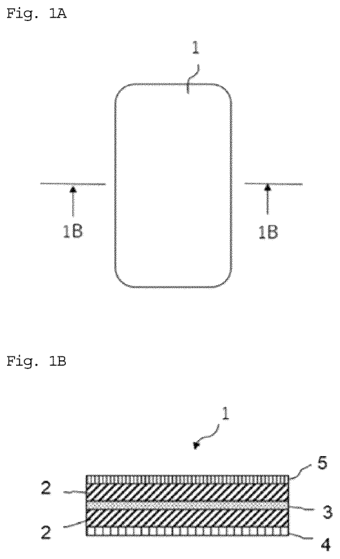

FIG. 1A is a schematic plan view of an embodiment of a gel sheet, and FIG. 1B is a cross-sectional view of the gel sheet of FIG. 1A taken along line 1B-1B. A gel sheet 1 includes a hydrogel 2 of the present invention, an intermediate substrate 3 is embedded along the in-plane direction of the hydrogel 2, and the intermediate substrate 3 is a nonwoven fabric or a woven fabric. In this embodiment, a base film 4 is provided on one side of the gel sheet 1, and a top film 5 is provided on a back side of the side on which the base film 4 is provided, but the base film 4 and the top film 5 may be omitted.

As the base film 4, for example, a resin film made of a resin such as polyester, polyolefin, polystyrene, or polyurethane, paper, paper obtained by laminating the resin film, or the like can be used.

The side of the base film 4 in contact with the gel sheet 1 has been preferably release-treated. Examples of the method for the release treatment include silicone coating, and the like, and in particular, baking-type silicone coating in which crosslinking and curing reactions are performed by heat or ultraviolet light is preferable. The film subjected to the release treatment is particularly preferably a biaxially stretched PET (polyethylene terephthalate) film, an OPP (stretched polypropylene) film, or the like.

The same material as that of the base film can be basically used for the top film 5. However, when polymerization is performed in a state in which the top film is provided by, for example, irradiating ultraviolet light from above the top film, it is preferable to select a film made of a material that does not block light so as not to interfere with photopolymerization.

### EXAMPLES

The present invention will be described in more detail using production examples and examples, but the present invention is not limited to these examples.

### Example 1

### 1. Preparation of Volatile Component-Containing Particle

As shown in Table 1, 1% by mass of l-menthol as a volatile component was dissolved in 1% by mass of NEOLIGHT 100P (manufactured by Kokyu Alcohol Kogyo Co., Ltd.) as a solvent using a stirring and mixing container to obtain a solvent solution of a volatile component. The solvent solution was added little by little to 2% by mass of particles A having parameters of an average pore diameter of 18 nm, an oil absorption of 150 ml/100 g, a pore volume of 0.40 ml/g, a specific surface area of 80 m²/g, a bulk specific gravity of 0.40 g/ml, and a volume average particle diameter of 8 μm, which were prepared with reference to our JP5812374B1, and after stirring the mixture, volatile component-containing particles were obtained.

### 2. Preparation of Mixture Liquid

As shown in Table 1, 20% by mass of acrylamide (AAM) as a monofunctional monomer, 0.040% by mass of N,N′-methylenebisacrylamide (MBAA) as a crosslinkable monomer, 54.8% by mass of glycerol as a wetting agent, and 18% by mass of ion exchange water, and 3.16% by mass of an electrolyte and a surfactant, a dispersant, a pH adjusting agent, an antiseptic agent, an initiator, a pressure-sensitive adhesive, and a chelating agent in total as other additives were added to a stirring and mixing container, and the mixture was stirred until completely dissolved to obtain a monomer solution. Thereafter, the volatile component-containing particles prepared in 1. were put into the monomer solution, and uniformly dispersed using a homomixer to obtain a mixture liquid.

### 3. Production of Hydrogel

Next, the obtained mixture liquid was dropped on a PET film (base film) with a thickness of 100 μm coated with silicone, and a nylon-based woven fabric and a PET film (top film) with a thickness of 38 μm coated with silicone were placed on the dropped mixture liquid to spread the mixture liquid uniformly, followed by fixing so that the thickness was 0.90 mm. Ultraviolet irradiation was performed to this mixture liquid in an energy amount of 3,000 mJ/cm² using a metal halide lamp, thereby obtaining a hydrogel sheet with a thickness of 0.90 mm of Example 1.

### Examples 2 to 10

Hydrogels of Examples 2 to 10 were produced in the same manner as in Example 1 except that the % by mass of each component was changed as shown in Table 1.

### Comparative Examples 1 to 4

Hydrogels of Comparative Examples 1 to 4 were produced in the same manner as in Example 1 except that the % by mass of each component was changed as shown in Table 1. In Comparative Examples 1, 2, and 4, the volatile component could not be stably dispersed and was precipitated in the hydrogel, and thus the hydrogel could not be produced.

Each component used in Examples 1 to 10 and Comparative Examples 1 to 4 is as follows.
AAM: Acrylamide
AA: Acrylic acid
DMAA: N,N-Dimethylacrylamide
MBAA: N,N′-Methylenebisacrylamide
l-Menthol: Menthol JP COS (manufactured by TAKASAGO INTERNATIONAL CORPORATION)
Lavender oil: LAVENDER GEL SK F 2263 (manufactured by INOUE PERFUMERY MFG. CO., LTD.)
Citrus oil: CITRUS GEL SK F 2264 (manufactured by INOUE PERFUMERY MFG. CO., LTD.)
NEOLIGHT 100P: Isodecyl neopentanoate (manufactured by Kokyu Alcohol Kogyo Co., Ltd.)
Particle A (porous resin particle): Average pore diameter: 18 nm, oil absorption: 150 ml/100 g, pore volume: 0.40 ml/g, specific surface area: 80 m²/g, bulk specific gravity: 0.40 g/ml, volume average particle diameter: 8.0 μm
Particle B (porous resin particle): Average pore diameter: 13 nm, oil absorption: 250 ml/100 g, pore volume: 0.70 ml/g, specific surface area: 200 m²/g, bulk specific gravity: 0.30 g/ml, volume average particle diameter: 8.0 μm
Particle C (porous resin particle): Average pore diameter: 18 nm, oil absorption: 100 ml/100 g, pore volume: 0.40 ml/g, specific surface area: 100 m²/g, bulk specific gravity: 0.60 g/ml, volume average particle diameter: 20 μm
Particles D (non-porous resin particle): Volume average particle diameter: 8.0 μm
SB-700 (porous inorganic particle): Porous silica particle, oil absorption: 400 to 700 ml/100 g, average particle diameter: 4.0 to 6.0 μm (manufactured by Miyoshi Kasei, Inc.)

### Evaluation of Odor Retention (Sustained Release)

After the hydrogel was peeled off from the PET film (base film), the odor of the gel surface after exposure to the atmosphere for 10 seconds was evaluated by 5 panels skilled in sensory test, and the score was obtained. Thereafter, a PET film (base film) was placed on the hydrogel, the same evaluation was performed 20 times, and the score was obtained each time. The average values of the evaluations of 5 persons in each of the first time, the tenth time, and the twentieth time were calculated. The results are shown in Table 1.

Score 1: Odorless

Score 2: The protective film was peeled off, and the nose was brought close to the gel surface to smell slightly.

Score 3: The odor was felt when the protective film was turned over.

Score 4: The odor was strongly felt when the protective film was turned over.

### Results

In the hydrogels of Examples 1 to 10, a large amount of volatile component was stably dispersed, and the sustained release of volatile component was good.

In the compositions of Comparative Examples 1, 2, and 4, the volatile component was precipitated in the hydrogel, and thus the hydrogel could not be produced. In the hydrogel of Comparative Example 3, the odor of the volatile component rapidly decreased, and the sustained release was poor.

TABLE 1

| | | | Example | Example | Example | Example | Example | Example | Example | Example |
|---|---|---|---|---|---|---|---|---|---|---|
| | | | 1 | 2 | 3 | 4 | 5 | 6 | 7 | 8 |
| (A) | Volatile component | I-Menthol | 1.0 | 2.0 | 2.0 | 2.0 | — | — | 2.0 | 2.0 |
| | (perfume) | | | | | | | | | |
| | Volatile component | Lavender oil | — | — | — | — | 1.5 | — | — | — |
| | (essential oil) | | | | | | | | | |
| | Volatile component | Citrus oil | — | — | — | — | — | 1.5 | — | — |
| | (essential oil) | | | | | | | | | |
| Solvent | | NEOLIGHT 100P | 1.0 | 2.0 | 2.0 | 2.0 | — | — | 2.0 | 2.0 |
| (B) | Porous resin | Particle A | 2.0 | 5.0 | — | — | 3.0 | 3.0 | 5.0 | 5.0 |
| | particle | Particle B | — | — | 5.0 | — | — | — | — | — |
| | | Particle C | — | — | — | 5.0 | — | — | — | — |
| | Non-porous | Particle D | — | — | — | — | — | — | — | — |
| | resin particle | | | | | | | | | |
| | Porous inorganic | SB-700 | — | — | — | — | — | — | — | — |
| | particle | | | | | | | | | |

| Monofunctional | AAM | 20 | 20 | 20 | 20 | 20 | 20 | 15 | 35 |
|---|---|---|---|---|---|---|---|---|---|
| monomer | AA | — | — | — | — | — | — | — | — |
| | DMAA | — | — | — | — | — | — | — | — |
| Crosslinkable monomer | MBAA | 0.040 | 0.040 | 0.040 | 0.040 | 0.040 | 0.040 | 0.030 | 0.070 |
| Wetting agent | Glycerol | 54.8 | 49.8 | 49.8 | 49.8 | 54.3 | 54.3 | 54.8 | 34.8 |
| Water | | 18 | 18 | 18 | 18 | 18 | 18 | 18 | 18 |
| Other additives | | 3.16 | 3.16 | 3.16 | 3.16 | 3.16 | 3.16 | 3.17 | 3.13 |
| Total | | 100 | 100 | 100 | 100 | 100 | 100 | 100 | 100 |
| Ratio (A):(B) | | 1:2 | 1:2.5 | 1:2.5 | 1:2.5 | 1:2 | 1:2 | 1:2.5 | 1:2.5 |
| Odor retention | First time | 3.4 | 3.8 | 3.6 | 3.6 | 3.6 | 3.2 | 3.6 | 3.4 |
| (Sustained release) | Tenth time | 3.2 | 3.2 | 3.6 | 3.6 | 3.4 | 3.0 | 3.2 | 3.2 |
| | Twentieth time | 2.8 | 3.0 | 3.2 | 3.2 | 2.8 | 3.0 | 2.8 | 3.0 |

| | | | Example | Example | Comparative | Comparative | Comparative | Comparative |
|---|---|---|---|---|---|---|---|---|
| | | | 9 | 10 | Example 1 | Example 2 | Example 3 | Example 4 |
| (A) | Volatile component | I-Menthol | 1.0 | 1.0 | 2.0 | 2.0 | 2.0 | 2.0 |
| | (perfume) | | | | | | | |
| | Volatile component | Lavender oil | — | — | — | — | — | — |
| | (essential oil) | | | | | | | |
| | Volatile component | Citrus oil | — | — | — | — | — | — |
| | (essential oil) | | | | | | | |
| Solvent | | NEOLIGHT 100P | 1.0 | 1.0 | 2.0 | 2.0 | 2.0 | 2.0 |
| (B) | Porous resin | Particle A | 2.0 | 2.0 | — | — | — | 0.80 |
| | particle | Particle B | — | — | — | — | — | — |
| | | Particle C | — | — | — | — | — | — |
| | Non-porous | Particle D | — | — | — | 4.0 | — | — |
| | resin particle | | | | | | | |
| | Porous inorganic | SB-700 | — | — | — | — | 4.0 | — |
| | particle | | | | | | | |

| Monofunctional | AAM | — | — | 20 | 20 | 20 | 20 |
|---|---|---|---|---|---|---|---|
| monomer | AA | 20 | — | — | — | — | — |
| | DMAA | — | 20 | — | — | — | — |
| Crosslinkable monomer | MBAA | 0.040 | 0.040 | 0.040 | 0.040 | 0.040 | 0.040 |
| Wetting agent | Glycerol | 54.8 | 54.8 | 54.8 | 50.8 | 50.8 | 54 |
| Water | | 18 | 18 | 18 | 18 | 18 | 18 |
| Other additives | | 3.16 | 3.16 | 3.16 | 3.16 | 3.16 | 3.16 |
| Total | | 100 | 100 | 100 | 100 | 100 | 100 |
| Ratio (A):(B) | | 1:2 | 1:2 | 1:0 | 1:2 | 1:2 | 1:0.4 |
| Odor retention | First time | 3.2 | 3.2 | Unproducible | Unproducible | 3.4 | Unproducible |
| (Sustained release) | Tenth time | 3.2 | 3.0 | | | 2.2 | |

| | | | |
|---|---|---|---|
| Twentieth time | 3.0 | 2.8 | 1.6 |

### DESCRIPTION OF REFERENCE NUMERALS

1 Gel sheet
2 Hydrogel
3 Intermediate substrate
4 Base film
5 Top film

1. A hydrogel comprising a porous resin particle and a volatile component,
the porous resin particle having a pore diameter of 5 to 30 nm and an oil absorption of 50 to 700 ml/100 g, and
a mass ratio between the volatile component (A) and the porous resin particle (B) contained in the hydrogel being
(A):(B)=1:1 to 1:4.

2. The hydrogel according to claim 1, wherein the porous resin particle comprises a monofunctional (meth)acrylic acid ester and a crosslinkable monomer.

3. The hydrogel according to claim 1, wherein a content of the volatile component is 0.5 to 5% by mass based on 100% by mass of the hydrogel.

4. The hydrogel according to claim 1, wherein the volatile component is at least one selected from the group consisting of perfumes, spices, and essential oils.

5. The hydrogel according to claim 1, comprising a polymer matrix, water, and a wetting agent.

6. The hydrogel according to claim 5, wherein the polymer matrix comprises a copolymer of a monofunctional monomer having one ethylenically unsaturated group, and a crosslinkable monomer.

7. The hydrogel according to claim 6, wherein a content of the structural unit derived from the monofunctional monomer is 10 to 40% by mass based on 100% by mass of the hydrogel.

8. The hydrogel according to claim 6, wherein the monofunctional monomer is at least one selected from the group consisting of (meth)acrylamide, (meth)acrylic acid, N,N-dimethyl (meth)acrylamide, diacetone (meth)acrylamide, and tert-butyl acrylamide sulfonic acid.

9. The hydrogel according to claim 5, wherein a content of the wetting agent is 20 to 70% by mass based on 100% by mass of the hydrogel.

10. The hydrogel according to claim 1, which is used as a biological electrode in a monitoring device or a device that performs treatment using electric stimulation, a counter electrode plate for an electrosurgical device, an adhesive tape, or a wound covering material.
